(19) United States
(12) Patent Application Publication
(10) Pub. No.: US 2014/0005584 A1
(43) Pub. Date: Jan. 2, 2014

(54) KNEE BRACE WITH EXPANSIBLE PRESSURE COMPONENTS

(71) Applicants: Ryan Russell Pretz, Poplar Bluff, MO (US); Clint Lee Vanlandingham, Poplar Bluff, MO (US)

(72) Inventors: Ryan Russell Pretz, Poplar Bluff, MO (US); Clint Lee Vanlandingham, Poplar Bluff, MO (US)

(21) Appl. No.: 13/833,265

(22) Filed: Mar. 15, 2013

Related U.S. Application Data
(60) Provisional application No. 61/690,603, filed on Jul. 2, 2012.
(60) Provisional application No. 61/743,295, filed on Aug. 31, 2012.
(60) Provisional application No. 61/743,296, filed on Aug. 31, 2012.
(60) Provisional application No. 61/743,277, filed on Aug. 31, 2012.
(60) Provisional application No. 61/795,886, filed on Oct. 31, 2012.

(51) Int. Cl. A61F 5/01

(52) U.S. Cl. CPC A61F 5/0123
USPC 602/13; 602/16

(57) ABSTRACT

A brace for treating knee contractures includes an inner lower arm and an outer lower arm. An arcuate strut connects the lower arms and superposes the posterior calf. The brace includes an inner upper arm and an outer upper arm. A second arcuate strut connects the upper arms and superposes the anterior thigh. A first lockable hinge connects the outer arms. A second hinge connects the inner arms. A first expansion means is situated on the first arcuate strut and is adapted to direct pressure from the strut to the calf. A second expansion means is situated on the second arcuate strut and is adapted to direct pressure from the strut to the front of the thigh. Once the one or more hinges are locked in therapeutic position, the expansion means can be expanded to provide a controlled and progressive additive stretch.

### CROSS REFERENCE TO RELATED APPLICATION

Not applicable.

### STATEMENT REGARDING FEDERALLY SPONSORED RESEARCH OR DEVELOPMENT

Not applicable.

### SEQUENCE LISTING, TABLE OR COMPUTER PROGRAM ON COMPACT DISC

Not applicable.

### FIELD OF INVENTION

This invention relates to knee brace devices designed to treat knee flexion contractures.

### BACKGROUND OF THE INVENTION

Knee flexion contracture is a condition of the lower leg wherein the knee is unable to fully straighten due to pain and stiffness. Limitations in knee extension range of motion are commonly treated with a prolonged, sustained and static stretch to the tissues producing the limitation of movement. Currently on the market there are a variety of devices intended to provide the static stretch to the lower leg at the knee joint. Although these devices are helpful, they have certain disadvantages.

FIG. 1A shows an exemplary prior art device used to treat knee flexion contractures. This device is a non-brace apparatus. It allows the knee to be forced into extension but it has limitations in that it does not provide the patient an option to incrementally progress through ranges of motion over time. Also, it requires that the patient situate him or herself in the device on the floor and therefore does not allow the patient to ambulate while using the device.

FIG. 1B shows an alternative prior art device used to treat knee flexion contractures. The device is a knee brace that holds the knee in a fixed position. Though the device allows the patient to wear the device while ambulating, the device does not provide for progressive range of motion adjustments.

FIG. 1C shows another prior art splinting device used to treat knee flexion contractures. This device is a dynamic splinting device that allows a patient to incrementally progress through ranges of knee extension. This device has noted drawbacks. In particular, this device has limitations in that it is difficult to use because the patient has to take the device off and utilize a screwdriver to adjust levels of tension.

### SUMMARY OF THE INVENTION

The present invention satisfies the need in the art and provides for a knee brace that is wearable and that easily and comfortably provides for progressive range of motion adjustments. The present invention satisfies this need by providing a knee brace that includes an inner lower rigid arm and an outer lower rigid arm. The lower rigid arms are respectively sized and spaced to fit along the inner (medial) and outer (lateral) sides of the lower leg of a user. The knee brace further includes a first rigid arcuate strut connecting the inner lower rigid arm to the outer lower rigid arm. The first rigid arcuate strut is shaped and located on the arms to superpose and fit on the posterior (calf area) lower leg of the user.

The present invention further includes an inner upper rigid arm and an outer upper rigid arm. These upper rigid arms are respectively sized and spaced to fit along the inner (medial) and outer (lateral) sides of the upper leg of a user. A second rigid arcuate strut connects the inner upper rigid arm to the outer upper rigid arm. This second rigid arcuate strut is shaped and located on the upper arms to superpose and fit around the anterior (quadriceps area) upper leg of the user.

A first lockable hinge operably connects the outer lower rigid arm to the outer upper rigid arm. This first hinge is located proximate to a knee of the user. A second hinge operably connects the inner lower rigid arm to the inner upper rigid arm. This second hinge may optionally be lockable. This second hinge is also located proximate to a knee of the user. A first expansion means is situated on the first rigid arcuate strut and is located so as to direct pressure from the strut to the posterior lower leg of the user. A second expansion means is situated on the second rigid arcuate strut and is located so as to direct pressure from the strut to the anterior upper leg of the user.

The knee brace can further include strapping or wrapping such that it can be secured to the upper and lower leg of a user and worn while walking. In the preferred embodiment one or more of the first and second expansion means provides for continuous expansion. This continuous expansion can be achieved with an inflatable bladder. Other types of continuous expansion means can be used to create pressure between the arcuate struts and the proximate leg portions. Such other continuous expansion means could include without limitation threaded cups that telescope upon each other by twisting movement or an eccentric rotatable cam.

In another embodiment the knee brace can further include a foot splint adapted to hold the foot of the user in a rigid position so as to treat conditions of the foot such as ankle contractures or plantar fasciitis. In one particular variant of this embodiment, the present invention knee brace can include a foot splint that is adapted to apply a dorsiflexion static stretch to the foot or toes of the user. Such splint can further include a third expansion means situated on the foot-bed of the splint that is adapted to direct pressure from the foot-bed to the bottom of foot to additively dorsiflex the foot or toes.

### BRIEF DESCRIPTION OF THE DRAWINGS

FIGS. 1A-1C show various prior art devices used to treat knee flexion contractures.

FIG. 2 is a front perspective view of a preferred embodiment knee brace of the present invention. For ease of view, the brace as shown does not include wrapping or straps such as would be used to secure the brace to the leg of a user while upright or walking.

FIG. 3 is a rear perspective view of a preferred embodiment knee brace of the present invention. For ease of view, the brace as shown does not include wrapping or straps such as would be used to secure the brace to the leg of a user while upright or walking.

FIG. 4 is a medial (inner leg) side elevation view of a preferred embodiment knee brace of the present invention. For ease of view, the brace as shown does not include wrapping or straps such as would be used to secure the brace to the leg of a user while upright or walking

FIG. 5 is a lateral (outer leg) side elevation view of a preferred embodiment knee brace of the present invention. For ease of view, the brace as shown does not include wrapping or straps such as would be used to secure the brace to the leg of a user while upright or walking.

FIG. 6 is a front elevation view of a preferred embodiment knee brace of the present invention. For ease of view, the brace as shown does not include wrapping or straps such as would be used to secure the brace to the leg of a user while upright or walking.

FIG. 7 is a rear elevation view of a preferred embodiment knee brace of the present invention. For ease of view, the brace as shown does not include wrapping or straps such as would be used to secure the brace to the leg of a user while upright or walking.

FIG. 8 is a front elevation view of a preferred embodiment upper rigid arm and lower rigid arm connected by a lockable hinge for use with the present invention knee brace.

FIG. 9 is a lateral side elevation view of a preferred embodiment upper rigid arm and lower rigid arm connected by a lockable hinge for use with the present invention knee brace.

FIG. 10 is a medial side elevation view of a preferred embodiment upper rigid arm and lower rigid arm connected by a lockable hinge for use with the present invention knee brace.

FIG. 11 is a front perspective view of a preferred embodiment knee brace of the present invention including a foot splint that is adapted to apply a static dorsiflexion stretch to the foot or toes of the user. For ease of view, the brace as shown does not include wrapping or straps such as would be used to secure the brace to the leg of a user while upright or walking.

FIG. 12 is a rear perspective view of a preferred embodiment knee brace of the present invention including a foot splint that is adapted to apply a dorsiflexion static stretch to the foot or toes of the user. For ease of view, the brace as shown does not include wrapping or straps such as would be used to secure the brace to the leg of a user while upright or walking.

FIG. 13 is a perspective view of the knee brace of the present invention with strapping attached so as to allow securement of the brace to the lower leg of a user.

### DETAILED DESCRIPTION

The preferred embodiment of the invention includes a static knee-locking splint to neutralize the knee into end-ranges of extension, with expansion means placed anterior to the femur and posterior to the tibia to allow the patient to provide incremental progression and intensity of knee extension stretch over time. The combination of the static splint and expansible components makes it easier to achieve the optimum amount of knee extension range of motion stretch for treatment because it is easier for the patient to don, doff and adjust than current devices on the market.

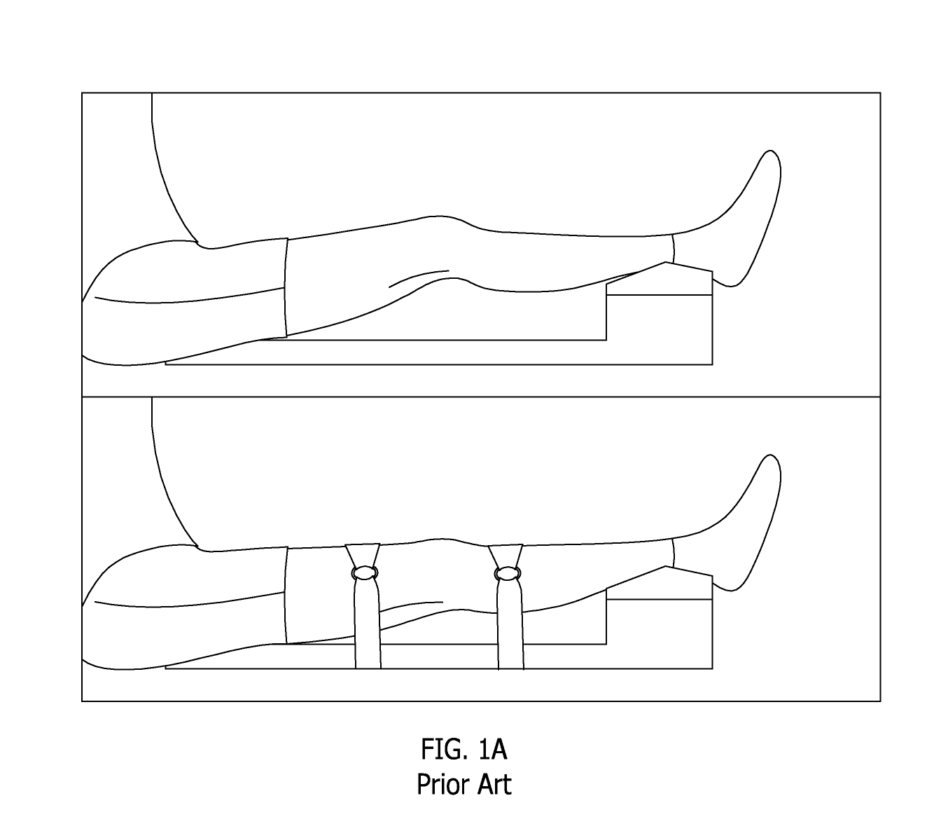

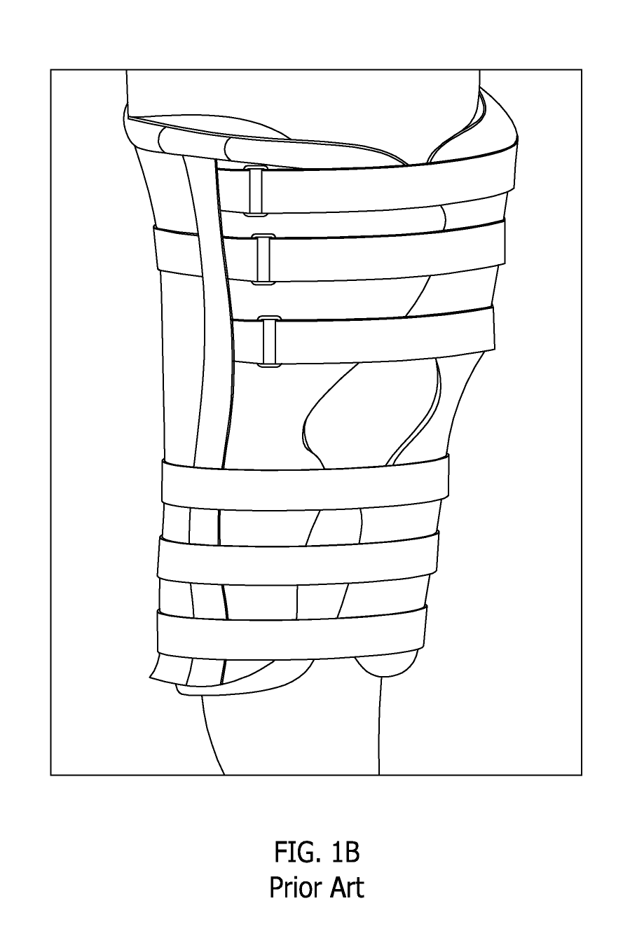

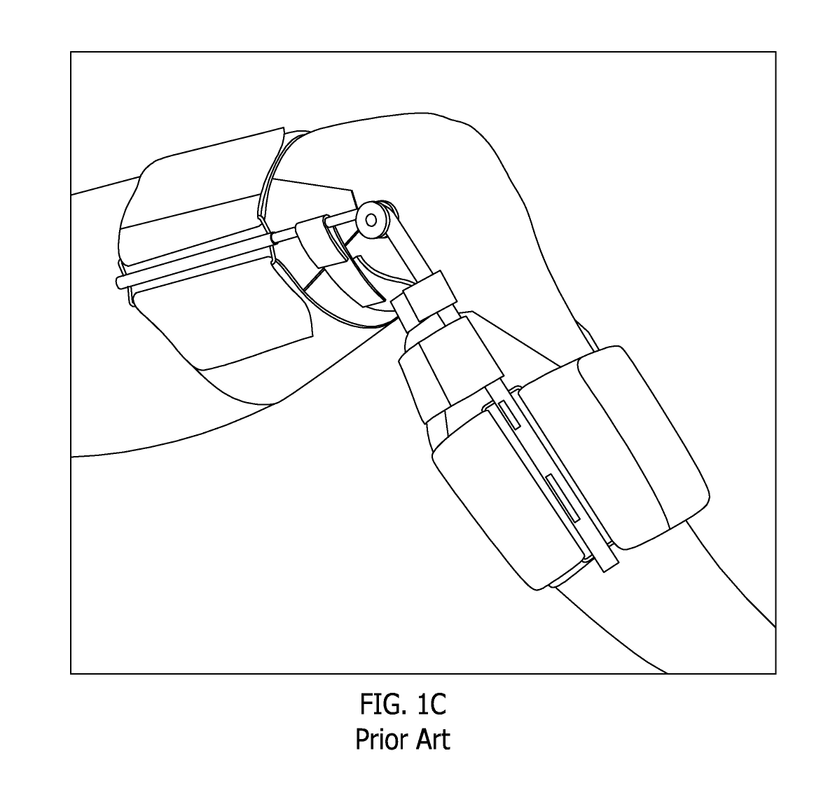

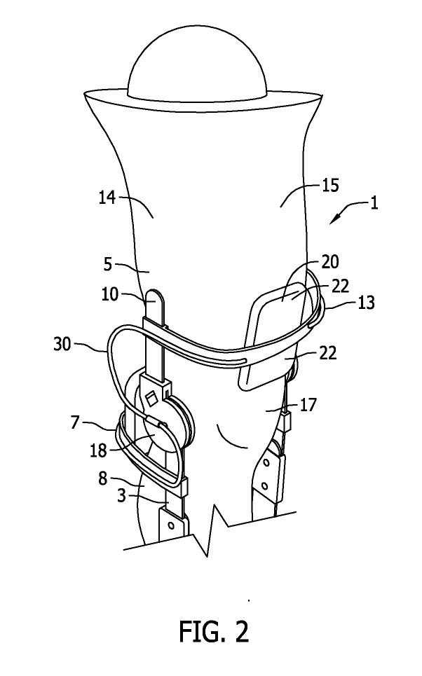

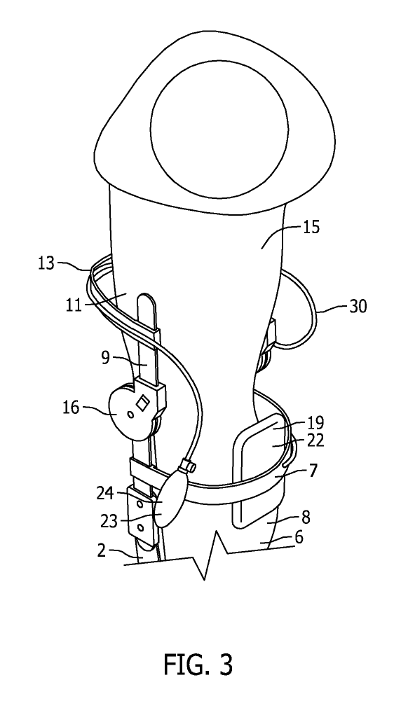

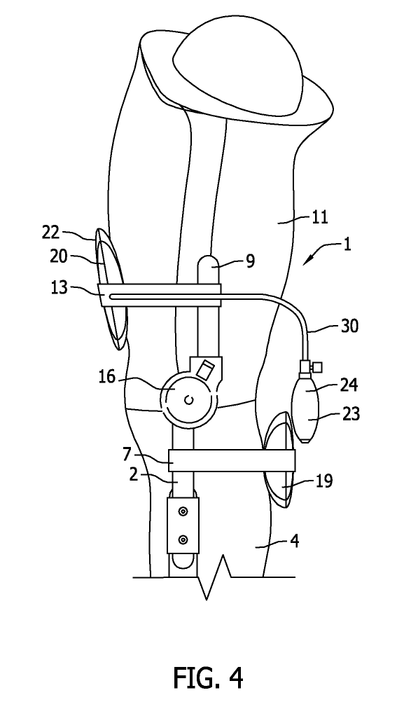

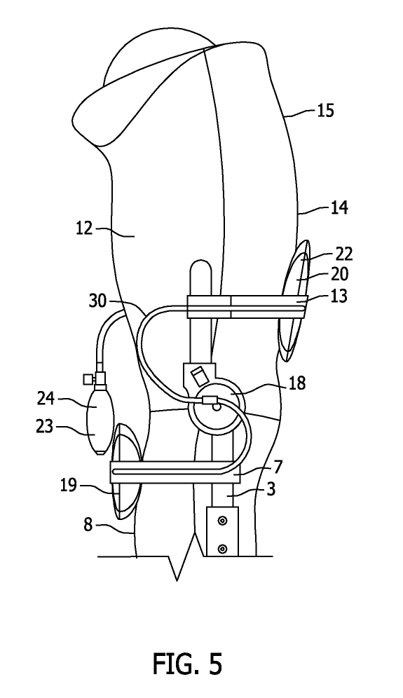

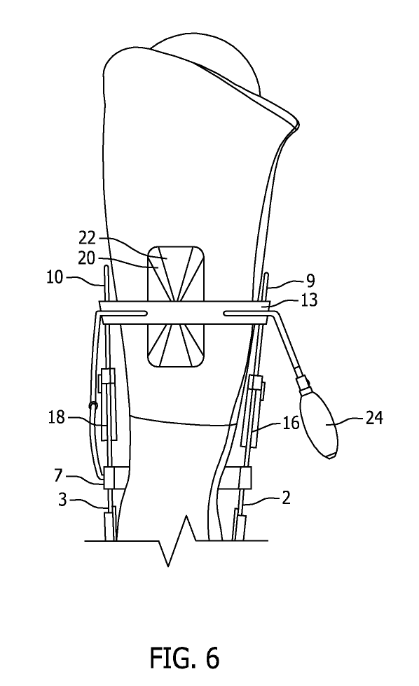

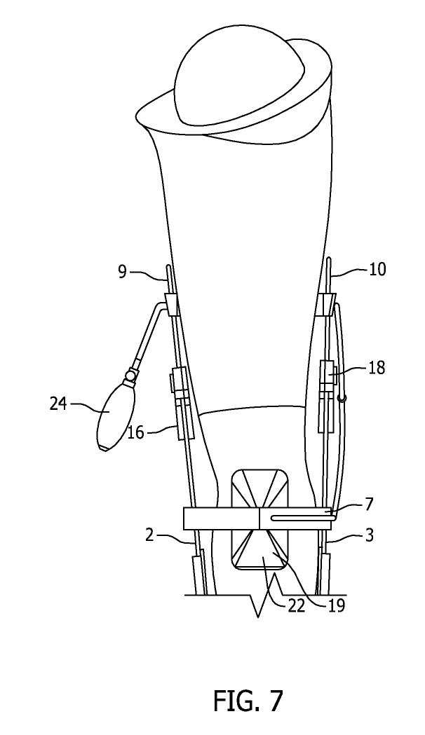

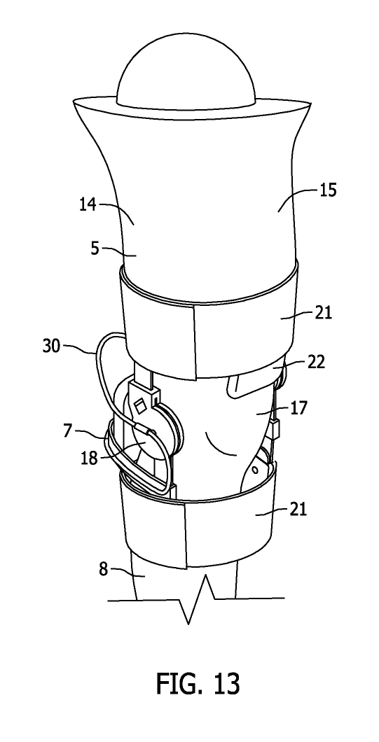

FIGS. 1-7 show an internal assembly of a preferred embodiment of the apparatus, with fabric strapping or wrapping removed so as to show the rigid structures of the brace. The brace with such suggested strapping or wrapping is shown in FIG. 13. Such covering is designed to achieve a comfortable frictional securement of the brace around the knee while the patient is standing or walking. Typical securement devices include straps with hook and loop fasteners (Velcro) or a fastenable neoprene sleeve.

As shown in the figures, the present invention knee brace 1 includes an inner lower rigid arm 2 and an outer lower rigid arm 3. The lower rigid arms 2, 3 are respectively sized and spaced to fit along the inner (medial) and outer (lateral) sides 4, 5 of the lower leg 6 of a user. Knee brace 1 further includes a first rigid arcuate strut 7 connecting inner lower rigid arm 2 to outer lower rigid arm 3. First rigid arcuate strut 7 is shaped and located on the arms 2, 3 to fit closely around the posterior (calf area) 8 of lower leg 6 of the user.

The present invention further includes an inner upper rigid arm 9 and an outer upper rigid arm 10. Upper rigid arms 9, 10 are respectively sized and spaced to fit along the inner (medial) and outer (lateral) sides 11, 12 of the upper leg 15 of a user. A second rigid arcuate strut 13 connects the inner upper rigid arm 9 to the outer upper rigid arm 10. Second rigid arcuate strut 13 is shaped and located on the upper arms 9, 10 to fit around the anterior (quadriceps area) 14 upper leg 15 of the user.

A first hinge 16 operably connects inner lower rigid arm 2 to inner upper rigid arm 9. First hinge 16 may be lockable and is located proximate to knee 17 of the user. A second lockable hinge 18 operably connects outer lower rigid arm 3 to the outer upper rigid arm 10. Second hinge 18 is also located proximate to knee 17 of the user. A first expansion means 19 is situated on first rigid arcuate strut 7 and is located so as to direct pressure from strut 7 to the posterior area 8 of lower leg 6 of the user. A second expansion means 20 is situated on second rigid arcuate strut 13 and is located so as to direct pressure from strut 13 to the anterior portion 14 of upper leg 15 of the user. Preferably, first expansion means 19 and second expansion means 20 are continuously expansible to apply smooth and better tolerated and controlled additive pressure. Such continuous expansion could be achieved via an inflatable bladder, which is the preferred embodiment expansion means. Optionally, expansion means could provide for incremental (indexed) expansion as well.

As shown in FIG. 13, knee brace 1 can further include strapping or wrapping 21 such that brace 1 can be secured to the upper leg 15 and lower leg 6 of a user and worn while walking. In the preferred embodiment one or more of the first and second expansion means 19, 20 can be an inflatable bladder 22. The depicted preferred embodiment knee brace 1 having inflatable bladder 22 can be inflated with any type of liquid or gas fluid. One such fluid could be ordinary room air. Accordingly, preferred embodiment knee brace with inflatable bladder 22 includes inflation means 23. Inflation means can comprise a bulb pump, a receptacle adapted to receive a pump needle or an inflation port with automatic sealing. In the shown preferred embodiment inflation means 23 is bulb pump 24 connected to bladder 22 by tubing 30. Bladders 22 can be made from molded rubber or other expandable or inflatable material. Bladders 22 are attached to arcuate struts 7 and 13 by any known method such as mechanical attachments or adhesives. In the preferred embodiment bladders 22 are attached to struts 7 or 13 by slots or a sleeve formed in the bladder 22 that receive the strut.

In addition to inflatable bladders, other types of continuous expansion means can be used to create pressure between the arcuate struts and the proximate leg portions 8, 14. Such other continuous expansion means could include without limitation threaded cups that telescope upon each other by twisting movement or an eccentric rotatable cam.

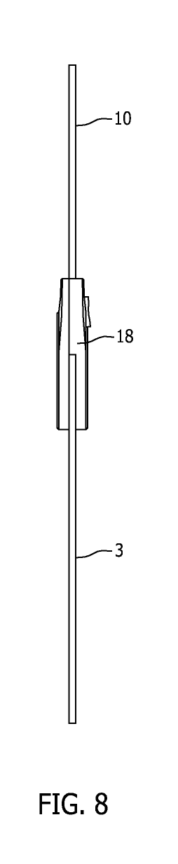

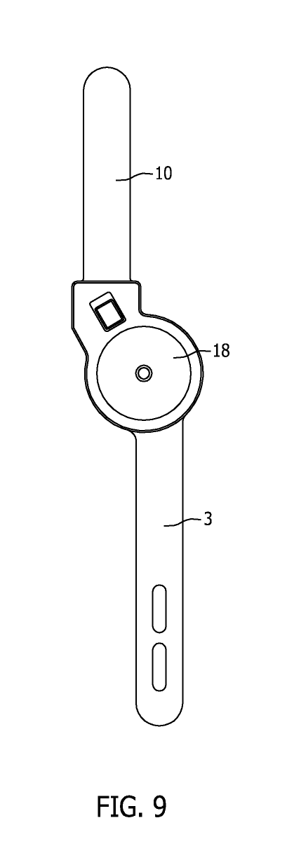

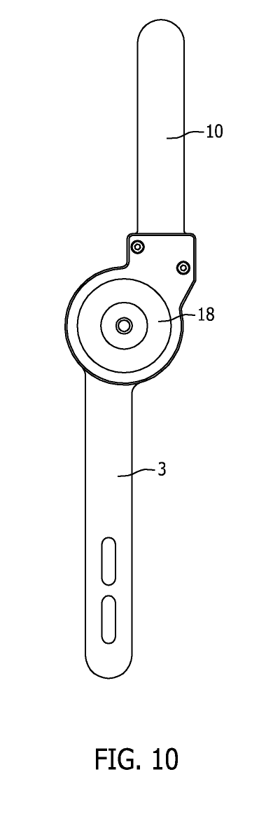

FIGS. 8, 9 and 10 show a preferred embodiment assembly of upper arms 9, 10 and lower arms 2, 3 connected by hinge 16, 18. These arms and hinge provide knee locking in any position (different ranges of motion) suitable to address the knee flexion contracture condition. One or more of hinges 16 and 18 includes any known locking feature to hold opposing upper arms 9, 10 respectively in position with lower arms 2, 3. Such a feature could be a friction lock, ratchet mechanism or a simple screw and internally threaded handle. Of key importance is that the hinge lock in full extension as dictated by the patient's range of motion.

In operation the user will typically attach knee brace 1 to the upper leg 15 and lower leg 6 thusly. The user will extend his or her knee to its maximum point of extension. The user will then lock hinges 16, 18 in position at this point so as to keep knee 17 in the extended position. At this point, the user can expand one or more of expansion means 19 and 20 to further increase the stretch at the knee joint. By expanding one or more of expansion means 19 and 20 an additive stretch is applied to the knee joint in a controlled, continuous and progressive fashion. This application of additional pressure causes increased stretching of the muscles and tissue responsible for knee flexion contracture. This type of increased stretching is much more comfortable for the user and easier to control. Likewise, reducing the stretch applied by knee brace 1 can be done easily and comfortably simply by collapsing one or more of expansion means 19 and 20. In the preferred embodiment knee brace, the additive stretch can be removed simply by deflating one or more of inflatable bladders 22.

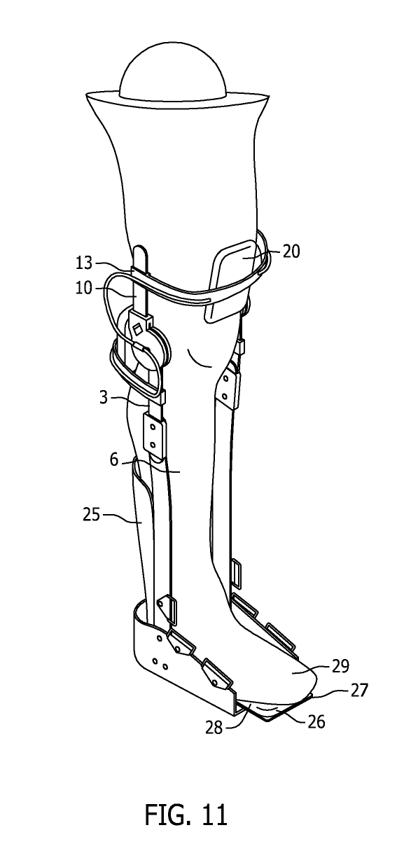

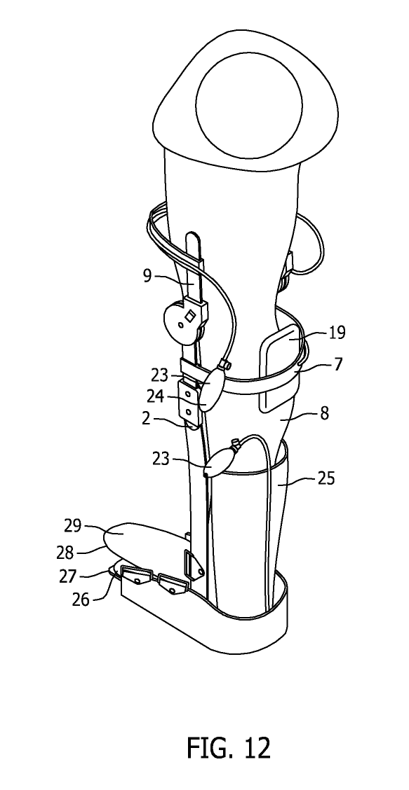

As shown in FIGS. 11 and 12, in another embodiment knee brace 1 can further include foot splint 25 adapted to hold the foot of the user in a rigid position so as to treat conditions of the foot such as ankle contractures or plantar fasciitis. In one particular variant of this embodiment, the present invention knee brace 1 can include foot splint 25 that is further adapted to apply a dorsiflexion static stretch to the foot or toes of the user. Foot splint 25 can be provided with a strap such that the user can pull back on the strap (not shown) to provide an additive stretch to the bicep femoris (hamstring) and the posterior lower leg muscles that attach above the knee. Such splint 25 can further or optionally include an underfoot wedge that is adapted to further dorsiflex the patient's toes and foot away from the foot-bed 27 on splint 25. In another embodiment, such splint 25 can further or optionally include a third expansion means 26 situated on the foot-bed 27 of splint 25 that is adapted to direct pressure from the foot-bed to the bottom 28 of foot 29. This pressure applies an additive stretch to the bottom 28 of foot 29 so as to increase the dorsiflexion stretch. of foot 29 in a controlled and progressive fashion. This application of additional pressure causes increased stretching of the foot, toes and muscles responsible for plantar fasciitis among other conditions.

The present invention further includes a method of treating lower leg and foot ailments due to compromised tissue elasticity. Such conditions include knee flexor contractures along with: degenerative conditions of the knee; internal knee derangement; patellofemoral syndrome; medial tibial stress syndrome; Charcot neuroarthropathy; gastroc equinus; calcaneal apophysiitis; foot/ankle arthriitis; ankle capsuliitis; achilles tendoniitis; posterior tibialis tendoniitis; anterior tibialis tendoniitis; plantar fasciitis; rheumatoid arthriitis; diabetic foot ulcers; forefoot capsulliitis; and metatarsalgia. The method of treatment would comprise providing the knee brace of any of the embodiments mentioned in this application. Next, the knee brace is placed on the leg of the patient such that the first expansion means is proximate the calf of the patient and the second expansion means is proximate the anterior thigh of the patient. At this point, either or both of the first and second expansion means are expanded such that one of the following actions occurs: a) the first expansion means applies pressure against the calf; b) the second expansion means applies pressure against the anterior thigh; or c) the first expansion means and the second expansion means respectively apply pressure against the calf and anterior thigh.

The present invention knee brace provides a novel device for treating knee flexion contractures. The device can be used on either the right or left leg without modifications. While particular embodiments of the present invention have been illustrated and described herein, the present invention is not limited to such illustrations and descriptions. The embodiments shown and described are merely preferred and alternative embodiments. It is apparent that changes and modifications may be incorporated and embodied as part of the present invention within the scope of the following claims.

What is claimed is:

1. A knee brace comprising:
an inner lower rigid arm and an outer lower rigid arm, the lower rigid arms respectively sized and spaced to fit along the inner and outer sides of the lower leg of a user;
a first rigid arcuate strut connecting the inner lower rigid arm to the outer lower rigid arm, the first rigid arcuate strut shaped and located to fit on the posterior lower leg of the user;
an inner upper rigid arm and an outer upper rigid arm, the upper rigid arms respectively sized and spaced to fit along the inner and outer sides of the upper leg of a user;
a second rigid arcuate strut connecting the inner upper rigid arm to the outer upper rigid arm, the second rigid arcuate strut shaped and located to fit on the anterior upper leg of the user;
a first hinge operably connecting the inner lower rigid arm to the inner upper rigid arm and located such that the first hinge is located proximate to a knee of the user;
a second hinge operably connecting the outer lower rigid arm to the outer upper rigid arm and located such that the second hinge is located proximate to a knee of the user;
the second hinge further being lockable;
a first expansion means situated on the first rigid arcuate strut and located so as to direct pressure from the strut to the posterior lower leg of the user and a second expansion means situated on the second rigid arcuate strut and located so as to direct pressure from the strut to the anterior upper leg of the user.

2. The knee brace of claim 1 further including strapping or wrapping to secure the brace to the upper and lower leg of a user.

3. The knee brace of claim 1 wherein one or more of the first and second expansion means is continuous.

4. The knee brace of claim 3 wherein one or more of the continuous expansion means is an inflatable bladder.

5. The knee brace of claim 4 further including inflation means, and the inflation means comprises one or more of a bulb pump, a receptacle adapted to receive a pump needle or an inflation port with automatic sealing.

6. The knee brace of claim 2 wherein one or more of the first and second expansion means is continuous.

7. The knee brace of claim 6 wherein one or more of the continuous expansion means is an inflatable bladder.

8. The knee brace of claim 7 further including inflation means, and the inflation means comprises one or more of a bulb pump, a receptacle adapted to receive a pump needle or an inflation port with automatic sealing.

9. The knee brace of claim 1 wherein the first hinge is lockable.

10. The knee brace of claim 1 further including a foot splint having a foot-bed adapted to receive the bottom of the foot of the user, the splint further adapted to hold the foot of the user in a rigid position.

11. The knee brace of claim 10 wherein the foot splint is further adapted to apply a dorsiflexion static stretch to the foot or toes of the user.

12. The knee brace of claim 11 wherein the application of dorsiflexion static stretch is provided through one or more of the following:
a) a strap attached to the foot splint;
b) a wedge between the foot of the user and the foot-bed; or
c) a third expansion means situated on the foot-bed and located so as to direct pressure from the foot bed to the bottom of the foot of the user.

13. The knee brace of claim 2 further including a foot splint having a foot-bed adapted to receive the bottom of the foot of the user, the splint further adapted to hold the foot of the user in a rigid position.

14. The knee brace of claim 13 wherein the foot splint is further adapted to apply a dorsiflexion static stretch to the foot or toes of the user.

15. The knee brace of claim 14 wherein the application of dorsiflexion static stretch is provided through one or more of the following:
a) a strap attached to the foot splint;
b) a wedge between the foot of the user and the foot-bed; or
c) a third expansion means situated on the foot-bed and located so as to direct pressure from the foot bed to the bottom of the foot of the user.

16. A method of treating lower leg and foot ailments in a patient due to compromised tissue elasticity, the method comprising:
providing the knee braces of any of claims 1 through 15,
placing the knee brace on the leg of the patient such that the first expansion means is proximate the calf of the patient and the second expansion means is proximate the anterior thigh of the patient; and
expanding either or both of the first and second expansion means such that one of the following actions occurs: a) the first expansion means applies pressure against the calf; b) the second expansion means applies pressure against the anterior thigh; or c) the first expansion means and the second expansion means respectively apply pressure against the calf and anterior thigh.

17. The method of claim 16 wherein the ailments include one or more of the following conditions: knee flexor contractures, degenerative conditions of the knee; internal knee derangement; patellofemoral syndrome; medial tibial stress syndrome; Charcot neuroarthropathy; gastroc equinus; calcaneal apophysiitis; foot/ankle arthriitis; ankle capsuliitis; achilles tendoniitis; posterior tibialis tendoniitis; anterior tibialis tendoniitis; plantar fasciitis; rheumatoid arthriitis; diabetic foot ulcers; forefoot capsulliitis; and metatarsalgia.
